(19) United States
(12) Patent Application Publication
(10) Pub. No.: US 2024/0350029 A1
(43) Pub. Date: Oct. 24, 2024

(54) NON-INVASIVE CARBON DIOXIDE SENSOR AND METHOD FOR MANUFACTURING THE SAME

(71) Applicant: National Tsing Hua University, Hsinchu City (TW)

(72) Inventors: Nyan-Hwa TAI, Hsinchu City (TW); Ya-Hsuan YANG, Hsinchu City (TW); Ching CHANG, Hsinchu City (TW); Chi-Young LEE, Hsinchu City (TW); Diahn-Warng PERNG, Taipei City (TW)

(21) Appl. No.: 18/640,090

(22) Filed: Apr. 19, 2024

(30) Foreign Application Priority Data
Apr. 24, 2023 (TW) 112115197

(51) Int. Cl. A61B 5/083; A61B 5/097; B82Y 30/00; B82Y 40/00

(52) U.S. Cl. CPC A61B 5/0836; A61B 5/097; A61B 2562/0285; A61B 2562/12; B82Y 30/00; B82Y 40/00

(57) ABSTRACT

A non-invasive carbon dioxide sensor includes a conductive substrate, a electrical transmission layer, and a gas sensing layer. The conductive substrate includes a base and at least two electrode disposed on the base and spaced apart from each other. The electrical transmission layer is disposed on the conductive substrate, and includes a plurality of carbon nanotubes crossing one another and a plurality of metal oxide nanorods attached to the carbon nanotubes. The carbon nanotubes and the metal oxide nanorods together form a composite material having a hierarchical three-dimensional structure. The gas sensing layer is disposed on the electrical transmission layer and includes a polymer material that contains at least one amino functional group capable of reacting with carbon dioxide. A method for manufacturing a non-invasive carbon dioxide sensor is also provided.

### CROSS-REFERENCE TO RELATED APPLICATION

This application claims priority to Taiwanese Invention Patent Application No. 112115197, filed on Apr. 24, 2023.

### FIELD

The disclosure relates to a carbon dioxide sensor and a method for manufacturing the same, and more particularly to a non-invasive carbon dioxide sensor and a method for manufacturing the same.

### BACKGROUND

Chronic obstructive pulmonary disease (COPD) is a progressive lung disease and is one of the highly prevalent as well as highly lethal diseases around the world. In addition, it is symptomized by shortness of breath and persistent cough due to low respiratory efficiency, which may in turn cause the accumulation of a large amount of carbon dioxide in a patient's body. Detection of carbon dioxide concentration in an individual's body is a commonly used method for determination whether or not the individual suffers from such disease. Nevertheless, nowadays most of the methods employed in clinic for detection carbon dioxide concentration are invasive and costly, and these shortcomings further cause a great decrease in the willingness of those who are likely to have a need.

Radial artery puncture, an invasive and well-known technique clinically used at present for detecting COPD, is conducted by collecting an arterial blood sample from a subject and then measuring the partial pressure of carbon dioxide therein. The subject undergone such invasive technique may suffer greater pain than that of one who is undergone a generous venous blood collection. Moreover, radial artery puncture is still a technique that must be conducted by appropriate professional medical personnel. In addition, there is yet another typically used technique performed by utilizing a capnography device, which can monitor carbon dioxide levels in the respiratory gases. However, this technique should be carried out under intubation, otherwise the measurement result will be affected by the carbon dioxide in the atmosphere, thus leading to a measurement error.

### SUMMARY

Therefore, an object of the disclosure is to provide a non-invasive carbon dioxide sensor and a method for manufacturing a non-invasive carbon dioxide sensor that can alleviate at least one of the drawbacks of the prior art.

According to a first aspect of the disclosure, the non-invasive carbon dioxide sensor includes a conductive substrate, an electrical transmission layer, and a gas sensing layer. The conductive substrate includes a base and at least two electrodes disposed on the base and spaced apart from each other. The electrical transmission layer is disposed on the conductive substrate and includes a plurality of carbon nanotubes crossing on another and a plurality of metal oxide nanorods attached to the carbon nanotubes, and the carbon nanotubes and the metal oxide nanorods together form a composite material having a three-dimensional structure. The gas sensing layer is disposed on the electrical transmission layer and includes a polymer material that contains at least one amino functional group capable of reacting with carbon dioxide.

According to a second aspect of the disclosure, the method for manufacturing a non-invasive carbon dioxide sensor includes:
(a) providing a conductive substrate including a base and at least two electrodes disposed on the base and spaced apart from each other;
(b) heating a composition containing a plurality of carbon nanotubes and a plurality of metal particles so as to transfer the composition into a composite containing a plurality of metal oxide particles attached to the carbon nanotubes;
(c) dispersing the composite obtained in step (b) in a solution containing a metal salt to form a mixture, followed by heating the mixture for a period of time to grow a plurality of metal oxide nanorods using the metal oxide particles attached to the carbon nanotubes as seeds, thereby obtaining an electrical transmission solution containing the carbon nanotubes and the metal oxide nanorods;
(d) heating the electrical transmission solution obtained in step (c);
(e) filtering the electrical transmission solution after step (d), thereby obtaining a composite material that includes the carbon nanotubes and the metal oxide nanorods and that has a three-dimensional structure;
(f) applying the composite material on the conductive substrate to form an electrical transmission layer on the conductive substrate; and
(g) applying a sensing solution on the electrical transmission layer and drying the sensing solution to form a gas sensing layer on the electrical transmission layer, the sensing solution including a polymer material containing at least one amino functional group.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a schematic view illustrating an embodiment of a non-invasive carbon dioxide sensor according to the disclosure.

FIG. 2 shows scanning electron microscope (SEM) images illustrating a surface morphology of carbon nanotubes without receiving an acid treatment.

FIG. 3 shows SEM images illustrating a surface morphology of acid-treated carbon nanotubes used in the embodiment of the non-invasive carbon dioxide sensor according to the disclosure.

FIG. 4 shows an SEM image and a corresponding atomic force microscope (AFM) image illustrating a morphology of a seed-ZnO-carbon nanotube composite (seed-ZCC).

FIG. 5 shows an SEM image and a corresponding AFM image illustrating a morphology of a composite material (0.5-ZCC) that contains carbon nanotubes and zinc oxide nanorods attached to the carbon nanotubes.

FIG. 6 shows an SEM image and a corresponding AFM image illustrating a morphology of a composite material (1-ZCC) that contains carbon nanotubes and zinc oxide nanorods attached to carbon nanotubes.

FIG. 7 shows an SEM image and a corresponding AFM image illustrating a morphology of a composite material (2-ZCC) that contains carbon nanotubes and zinc oxide nanorods attached to carbon nanotubes.

FIG. 8 is a schematic view illustrating a measuring system including the embodiment of the non-invasive carbon dioxide sensor according to the disclosure.

FIG. 9 is a bar graph showing response values determined in 1PEI, 2PEI, and 3PEI.

FIG. 10 is a bar graph showing response values determined in four of the non-invasive carbon dioxide sensors according to the disclosure having electrical transmission layers prepared from seed-ZCC, 0.5-ZCC, 1-ZCC, and 2-ZCC, respectively.

FIG. 11 is a bar graph showing response values determined in 10-1-ZCC, 30-1-ZCC, 50-1-ZCC, 70-1-ZCC, and 90-1-ZCC.

FIG. 12 is line graph showing the relationship between response values and carbon dioxide concentrations under different relative humidities.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

It should be noted herein that for clarity of description, spatially relative terms such as “top,” “bottom,” “upper,” “lower,” “on,” “above,” “over,” “downwardly,” “upwardly” and the like may be used throughout the disclosure while making reference to the features as illustrated in the drawings. The features may be oriented differently (e.g., rotated 90 degrees or at other orientations) and the spatially relative terms used herein may be interpreted accordingly.

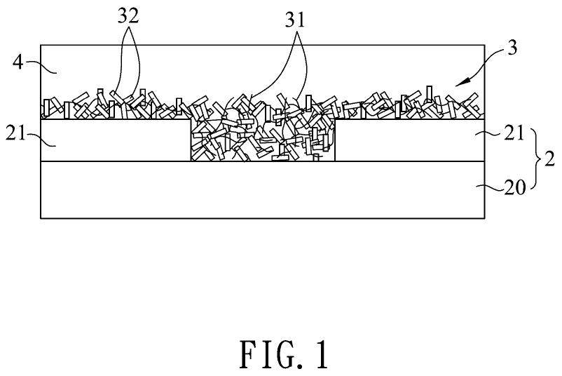

Referring to FIG. 1, an embodiment of a non-invasive carbon dioxide sensor according to the disclosure is adapted to detect a carbon dioxide concentration in a respiratory gas (e.g., exhale breath) of a subject to be tested at room temperature, and includes a conductive substrate 2, an electrical transmission layer 3 disposed on the conductive substrate 2, and a gas sensing layer 4 disposed on the electrical transmission layer 3.

To illustrate, the conductive substrate 2 includes a base 20 and at least two electrodes 21 disposed on the base 20 and spaced apart from each other. In certain embodiments, the base 20 may be a fluorine-doped tin oxide (FTO) substrate, an indium tin oxide (ITO) substrate, a silicon (Si) substrate, a glassy carbon substrate or a metal substrate. In certain embodiments, the base 20 may be a polypropylene (PP) base, a polyimide (PI) flexible base, a polyethylene terephthalate (PET) flexible base, a carbon base, or an ultra-thin glass base. Indeed, the form and type of the electrodes 21 are not so limited as long as they can be properly set on the base 20. In this embodiment, the conductive substrate 2 is exemplified by a PP base printed with carbon electrodes located at interval for illustration. Incidentally, the conductive substrate 2 is not a focus of the disclosure and will not be detailed described herein.

The electrical transmission layer 3 includes a plurality of carbon nanotubes (CNTs) 31 randomly crossing one another and a plurality of metal oxide nanorods 32 randomly attached to the carbon nanotubes 31, and the carbon nanotubes 31 and the metal oxide nanorods 32 together form a composite material having a three-dimensional structure. The composite material may have a hierarchical three-dimensional structure. As used herein, the “hierarchical three-dimensional structure” refers to a structure having an integrated architecture composed of different dimensional components, which may include a zero-dimensional component (e.g., a dot), a one-dimensional component (e.g., a wire or a line), and a two-dimensional component (e.g., a layer). The carbon nanotubes 31 may be functionalized carbon nanotubes with functional groups such as hydroxyl groups or carboxyl groups. In certain embodiments, the metal oxide nanorods 32 may be zinc oxide (ZnO) nanorods, titanium dioxide (TiO₂) nanorods, aluminum oxide (Al₂O₃) nanorods, tricobalt tetroxide (Co₃O₄) nanorods, tin dioxide (SnO₂) nanorods, copper oxide (CuO) nanorods, zirconium dioxide (ZrO₂) nanorods, or combinations thereof. In this embodiment, ZnO nanorods are utilized as an example for illustration, that is to say, the electrical transmission layer 3 of this embodiment is formed by the ZnO nanorods and the carbon nanotubes 31. For ease of description, the electrical transmission layer 3 formed of the composite material having the ZnO nanorods and the carbon nanotubes 31 will be abbreviated as “ZCC” in following narrative as well as in the Figures. In other embodiments, the electrical transmission layer 3 may have a single layer structure or a multilayer structure. In still other embodiments, the non-invasive carbon dioxide sensor may include X number of the electrical transmission layers 3, where 10≤X≤90.

The gas sensing layer 4 includes a polymer material that contains at least one amino functional group capable of reacting with carbon dioxide, that is, the main function of the gas sensing layer 4 is to capture carbon dioxide. Accordingly, the polymer material used for forming the gas sensing layer 4 may be varied as long as having such amino functional group for capturing carbon dioxide. Additionally, in some embodiments, the gas sensing layer 4 may be of a single layer structure or a multilayer structure. In still some other embodiments, the non-invasive carbon dioxide sensor may include Y number of the gas sensing layers 4, where 1≤Y≤6. In certain embodiments, the polymer material may be polyethylenimine (PEI), polypyrrole (PPy), polyaniline (PANI), or combinations thereof. In this embodiment, PEI serving as the polymer material is utilized as an example for illustration.

In certain embodiments, the carbon nanotubes 31 may be acid-treated carbon nanotubes 31.

The non-invasive carbon dioxide sensor according to the disclosure of this embodiment may be manufactured by a method, which may include the following steps (a) to (g).

In step (a), the conductive substrate 2 including the base 20 and the at least two electrodes 21 disposed on the base 20 and spaced apart from each other is provided. Specifically, the conductive substrate 2 is initially subjected to an oxygen plasma treatment so as to make a surface thereof more hydrophilic, thereby enhancing the adherence between the surface and materials to be subsequently disposed thereon.

In step (b′), the carbon nanotubes 31 is subjected to an acid treatment so as to efficiently improve the hydrophilic property thereof. To illustrate, the acid treatment is conducted by adding the carbon nanotubes 31 into an acid solution containing nitric acid (HNO₃) and sulfuric acid (H₂SO₄), and heating the acid solution along with the carbon nanotubes 31 to a temperature ranging from 70° C. to 100° C. for a predetermined time period. In addition, in this step, the acid treatment may further includes cooling the acid solution, followed by rinsing the carbon nanotubes 31 with deionized water (DI water) to neutralize it, and drying the carbon nanotubes 31, so as to obtain acid-treated carbon nanotubes 31. In this embodiment, the acid treatment is conducted by adding the carbon nanotubes 31 into an acid solution containing 14 M nitric acid and 18 M sulfuric acid in a volume ratio of 1:3, heating the acid solution along with the carbon nanotubes 31 to a temperature of 100° C. for 2 hours, followed by rinsing the carbon nanotubes 31 with deionized water to neutralize it, vacuum filtrating the acid solution to obtain the carbon nanotubes 31, and then drying the carbon nanotubes 31, so as to obtain the acid-treated carbon nanotubes 31. The purpose of the acid treatment in step (b′) is to make surfaces of the carbon nanotubes 31 have higher hydrophilicity so as to enhance the nucleation of metal oxide particles and the growth of the metal oxide nanotubes 32 (will be mentioned below).

Additionally, hydrophilicity of the surface of the conductive substrate 2 of this embodiment is assessed by water contact angle (WCA) analysis, both before and after the oxygen plasma treatment. The results show that a water contact angle of the surface of the conductive substrate 2 before the oxygen plasma treatment is 124.85° and a water contact angle of the surface of the conductive substrate 2 after the oxygen plasma treatment is 4.66°, validating that the oxygen plasma treatment can provide the surface of the conductive substrate 2 with excellent hydrophilicity.

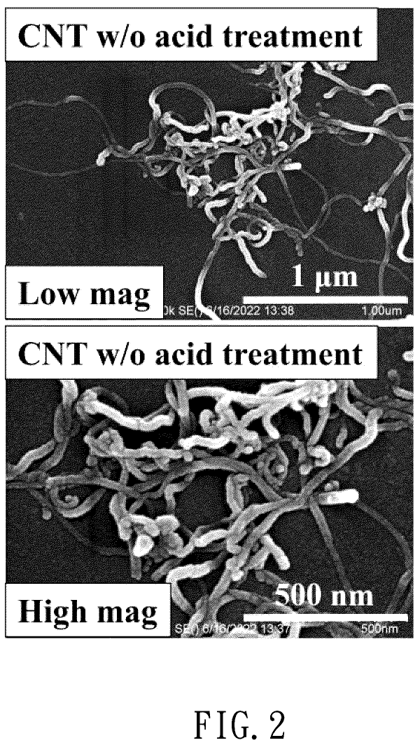

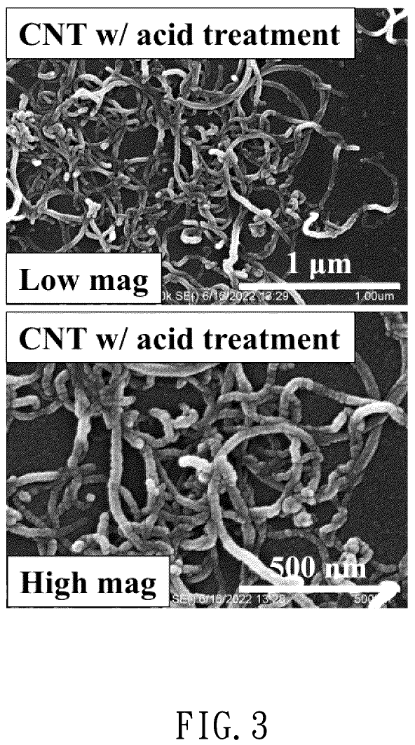

In addition, referring to FIGS. 2 and 3, before the acid treatment (as shown in FIG. 2), the surface of the carbon nanotubes 31 shows few defects; in contrast, after the acid treatment (as shown in FIG. 3), defects thereon are much increased, and such outcome will greatly aid the implementation of step (b) afterwards. Furthermore, the carbon nanotubes 31 of this embodiment are subjected to Raman analysis as well as Fourier transform infrared spectroscopy (FTIR) analysis, both before and after the acid treatment. For brief description of these two analyses, only a short account with respect to the results thereof may be represented as follows. The results of the Raman analysis show that, before the acid treatment, the carbon nanotubes 31 has an intensity ratio of D to G bands (ID/IG ratio) of merely 1.44; however, after the acid treatment, the acid-treated carbon nanotubes 31 has an ID/IG ratio of up to 1.91, revealing that, after the acid treatment, the defects on the carbon nanotubes 31 are significantly increased. Moreover, the results of the Fourier transform infrared spectroscopy analysis show that, after the acid treatment, the carbon nanotubes 31 can allow oxygen-containing hydrophilic functional groups to be grafted on the surfaces thereof. Consequently, the surface of the acid-treated carbon nanotubes 31 may have high hydrophilicity and hence the acid-treated carbon nanotubes 31 exhibit good dispersibility in an aqueous solution.

In step (b), a composition containing a plurality of the acid-treated carbon nanotubes 31 and a plurality of metal particles is heated so as to form the composition into a composite containing a plurality of the metal oxide particles attached to the acid-treated carbon nanotubes 31. In addition, the metal oxide particles are obtained by oxidizing the metal particles and function as seeds. Specifically, the metal oxide particles function as seeds for growth of the metal oxide nanorods 32. In this embodiment, the step (b) is conducted by heating the composition containing the acid-treated carbon nanotubes 31 and zinc particles (serving as the metal particles) in a furnace tube at 800° C. for 1 hour, so as to transfer the composition into the composite containing zinc oxide particles (ZnO) (serving as the metal oxide particles) attached to the acid-treated carbon nanotubes 31. Hereinafter, the composite is referred to as seed-ZCC.

In step (c), the composite obtained in step (b) is dispersed in a solution (containing a metal salt and a solvent) to form a mixture, followed by heating the mixture for a period of time to grow a plurality of the metal oxide nanorods 32 from the metal oxide particles attached to the acid-treated carbon nanotubes 31, thereby obtaining an electrical transmission solution containing the acid-treated carbon nanotubes 31, the metal oxide nanorods 32, and a byproduct. In certain embodiments, heating of the mixture may be conducted in a water bath. In certain embodiments, the metal salt may be a zinc salt, a titanium salt, an aluminum salt, a cobalt salt, a tin salt, a copper salt, or a zirconium salt. In an exemplary embodiment, the metal salt is the zinc salt. In other embodiments, the solution of this step may further contain a solvent and a stabilizing agent. In this embodiment, the zinc salt is zinc nitrate (Zn(NO₃)₂), the stabilizing agent is hexamethylenetetramine ((CH₂)₆N₄; HMTA), and the solvent is deionized water. In an example, three of the aforesaid composites (seed-ZCCs) were prepared, and each of the three composites was added into a solution containing 0.02 M of zinc nitrate, 0.05 M of hexamethylenetetramine, and 100 ml of deionized water to form a mixture. The three mixtures were heated for 0.5 hours, 1 hour, and 2 hours, respectively, so as to grow zinc oxide nanorods 32 from the zinc oxide particles attached to the acid-treated carbon nanotubes 31 through the supplement of zinc ions in the solution, thereby obtaining three electrical transmission solutions each containing the acid-treated nanotubes 31, the zinc oxide nanorods 32, and a byproduct (i.e., impurities). The zinc oxide nanorods 32 in the three electrical transmission solutions have different lengths due to different heating times. Hereinafter, the composite material containing the acid-treated nanotubes 31 and the zinc oxide nanorods 32 in the electrical transmission solution that was heated for 0.5 hours is referred to as 0.5-ZCC, the composite material containing the acid-treated nanotubes 31 and the zinc oxide nanorods 32 in the electrical transmission solution that was heated for 1 hour is referred to as 1-ZCC, and the composite material containing the acid-treated nanotubes 31 and the zinc oxide nanorods 32 in the electrical transmission solution that was heated for 2 hours is referred to as 2-ZCC (heated for 2 hour). It should be noted that the metal oxide nanorods 32, i.e., the zinc oxide nanorods, may essentially serve a function to expand space within the acid-treated carbon nanotubes 31 to generate the hierarchical three-dimensional structure. In addition, the three-dimensional structure formed by the acid-treated carbon nanotubes 31 and the metal oxide nanorods 32 can not only let the electrical transmission layer 3 attain higher conductivity but also provide a greater surface area so as to have a good contact with the gas sensing layer 4.

In step (d), each of the electrical transmission solutions was further heated, and then, in step (e), was filtrated so as to remove the byproduct, thereby obtaining the composite materials each having the hierarchical three-dimensional structure. In certain embodiments, step (e) may conducted by vacuum filtration.

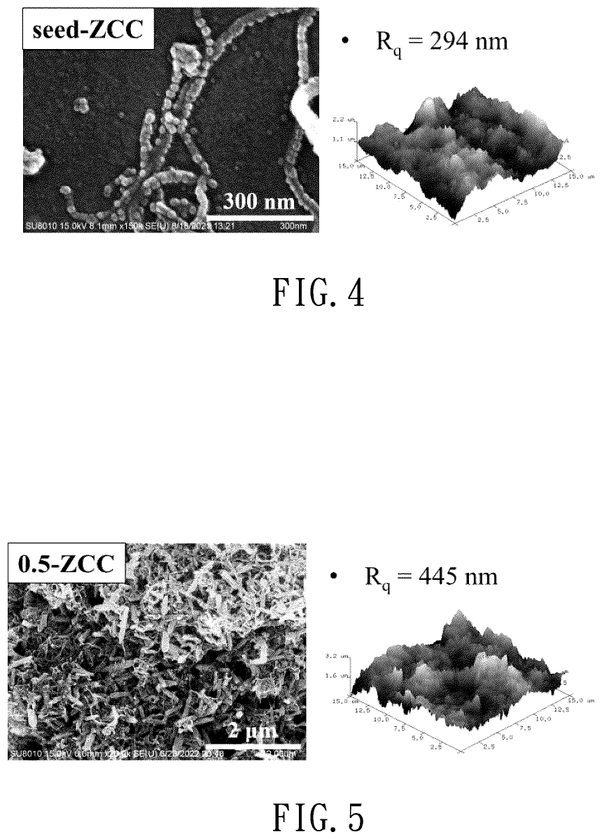

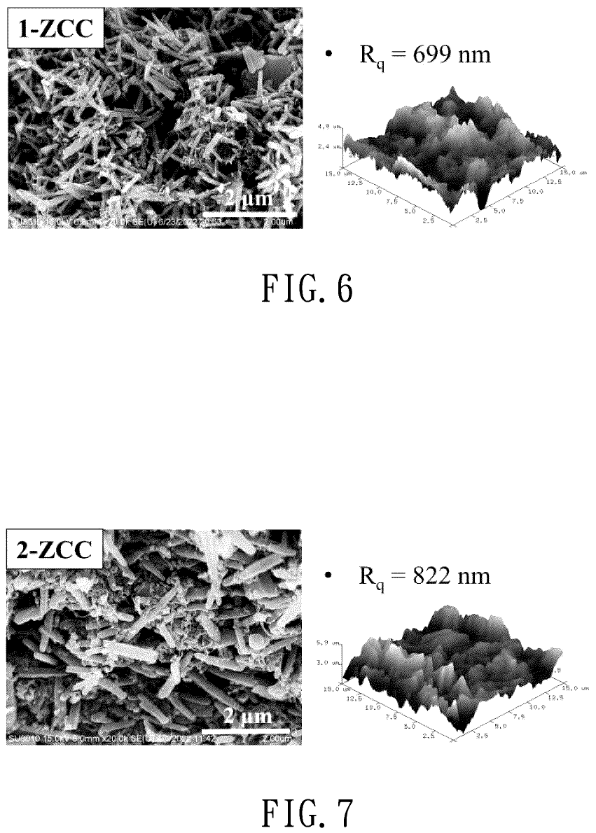

Referring to FIGS. 4 to 7, it can be seen that as the heating time for the growth of the zinc oxide nanorods 32 from the zinc oxide particles increases, the surface roughness of the acid-treated carbon nanotubes 31 with the metal oxide particles/metal oxide nanorods 32 formed thereon increases accordingly (i.e., Rq being increased from 294 nm to 822 nm), which may be beneficial to the increase of surface area of the gas sensing layer 4 later.

In step (f), the composite material is uniformly applied, e.g., coated on the conductive substrate 2 to form the electrical transmission layer 3 on the conductive substrate 2. To be specifically, the composite material is added into a solvent to form a mixture. The mixture is then coated on the conductive substrate 2 to form the electrical transmission layer 3 on the conductive substrate 2. In some embodiments, step (f) may be applied repeatedly for several times.

In an example, a plurality of the conductive substrates 2 were provided, and step (f) was conducted by mixing each of the seed-ZCC obtained in step (b) after vacuum filtration and the composite materials (0.5-ZCC, 1-ZCC, or 2-ZCC) obtained in step (d) with 100 ml of isopropanol (IPA, as the solvent in step (f)), so as to form a mixture, followed by drop casting the mixture on a respective one of the conductive substrates 2 to form the electrical transmission layer 3 thereon. For each of the composite materials, step (f) was conducted repeatedly for 10 times, 30 times, 50 times, 70 times, and 90 times on the conductive substrates 2, respectively, so as to obtain samples with 10, 30, 50, 70, and 90 of the electrical transmission layers 3.

In step (g), a sensing solution is coated on the electrical transmission layer 3, and then is dried to form the gas sensing layer 4 on the electrical transmission layer 3. The sensing solution includes the polymer material having the amino functional group. In certain embodiments, step (g) is conducted repeatedly for several times to form a plurality of the gas sensing layers 4 on the electrical transmission layer 3, and the latter applying process of the sensing solution is conducted until the sensing solution previously coated is dried to form the gas sensing layer 4. In an example, the sensing solution was obtained by dissolving polyethyleneimine (PEI) in IPA, and was spray coated on the electrical transmission layer 3 for one, two or three times, thereby gaining one, two or three of the gas sensing layers 4 on the electrical transmission layers 3, and thus the non-invasive carbon dioxide sensor according to the disclosure is obtained.

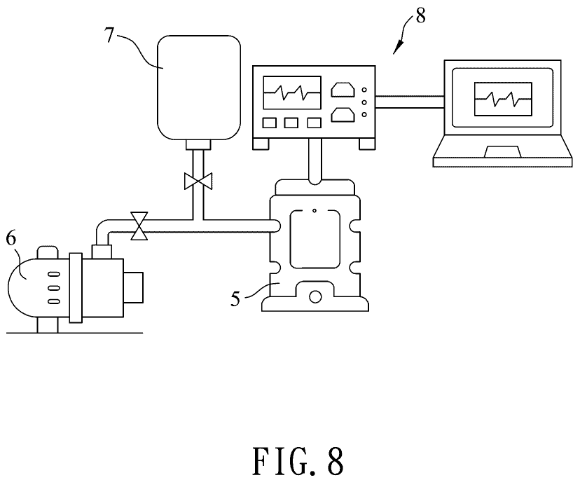

The non-invasive carbon dioxide sensors manufacturing by the aforementioned method according to the disclosure are applied to a measuring system as shown in FIG. 8. The measuring system includes a chamber 5 for accommodating one of the non-invasive carbon dioxide sensor therein, an air extracting pump 6 and an air collecting bag 7 both spatially communicating with the chamber 5, and a power supply unit 8 electrically connected to the non-invasive carbon dioxide sensors located in the chamber 5.

While carrying out a measurement, initially, one of the non-invasive carbon dioxide sensors is placed in the chamber 5, and then gas in the chamber 5 surrounding the non-invasive carbon dioxide sensor is extracted using the air extracting pump 6. After that, a subject to be tested exhales a breath into the air collecting bag 7, and the exhaled breath then moves to the chamber 5 so that carbon dioxide in the exhaled breath is captured by the non-invasive carbon dioxide sensor and reacted with the polymer of the non-invasive carbon dioxide sensor. Afterward, a current value in the non-invasive carbon dioxide sensor can be measured by the power supply unit 8, thereby calculating a response value of the non-invasive carbon dioxide sensor.

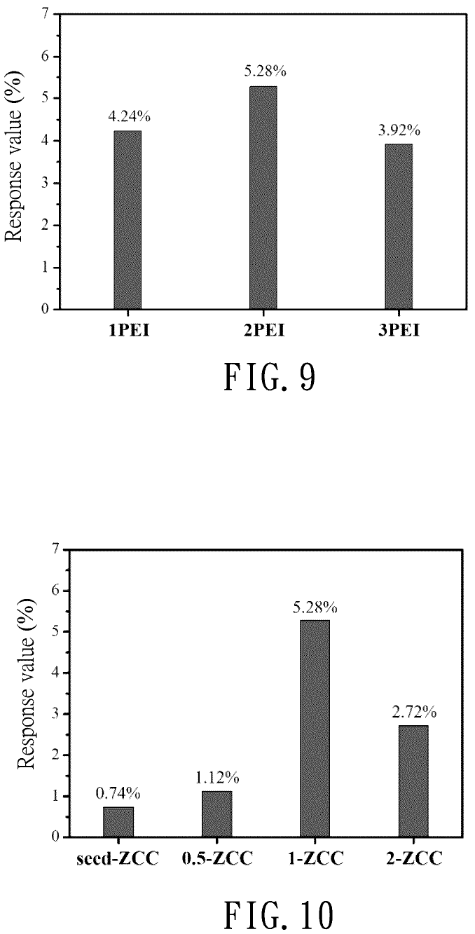

Referring to FIG. 9, “1PEI”, “2PEI” and “3PEI” therein respectively represent the non-invasive carbon dioxide sensors having one, two and three of the gas sensing layers 4. Each of the non-invasive carbon dioxide sensors with different number of the gas sensing layers 4 is subjected to the aforementioned measurement for determining its response value. It can be seen from FIG. 9 that the non-invasive carbon dioxide sensor containing two gas sensing layers 4 has the highest response value (5.28%).

The non-invasive carbon dioxide sensors respectively having seed-ZCC, 0.5-ZCC, 1-ZCC, and 2-ZCC are subjected to the aforesaid measurement for determining their response values. Referring to FIG. 10, the non-invasive carbon dioxide sensor having 1-ZCC has the highest response value (5.28%). However, the response value of the non-invasive carbon dioxide sensor having 2-ZCC drops to 2.72% due to a decreased current value caused by an excessive amount of electrically non-conductive zinc oxide.

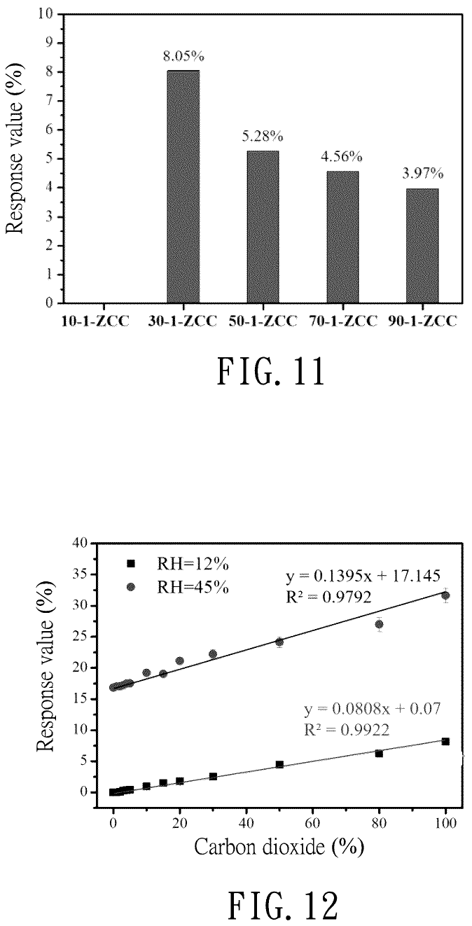

Referring to FIG. 11, it should be noted that “30-1-ZCC” therein represents that the non-invasive carbon dioxide sensor has 30 of the electrical transmission layers 3 and 1-ZCC is used as the composite material of the electrical transmission layers 3. The meanings of “50-1-ZCC”, “70-1-ZCC”, and “90-1-ZCC” can also be deduced in the same way. It can be seen that the non-invasive carbon dioxide sensor having 30 of the electrical transmission layers 3 has the highest response value (8.05%) due to possessing the greatest surface area (provided by 30 of the electrical transmission layers 3). The synergistic effect between gas sensing layer 4 and the hierarchical three-dimensional structure of the electrical transmission layer 3 effectively enhances the surface roughness of the electrodes 21, thereby increasing the contacting surface of the gas sensing layer 4 (i.e., the PEI coating) with the electrical transmission layers 3 and hence increasing the response value.

In addition, the non-invasive carbon dioxide sensor according to the disclosure exhibits a significant difference performance in measuring response value under different circumstances with different humidities. Therefore, the 30-1-ZCC, which shows the highest response value, is further subjected to measurements to determine response values under different carbon dioxide concentrations and different relative humidities (RH), i.e., RH 12% and RH 45%. Referring to FIG. 12, it can be seem that the relationship between the response value and the carbon dioxide concentration is positively correlated, both at RH 12% and at RH 45%, confirming that the non-invasive carbon dioxide sensor according to the disclosure presents an excellent resolution capacity in sensing carbon dioxide with stable reproducibility.

In sum, the electrical transmission layer 3 made of the composite material including the carbon nanotubes 31 and the metal oxide nanorods 32 possesses the hierarchical there-dimensional structure on the conductive substrate 2, and the subsequently formed gas sensing layer 4 could be effectively adhered on the electrical transmission layer 3, so as to perform good electrical conductivity. The non-invasive carbon dioxide sensor can be used at room temperature, and can indeed achieve the goal of providing a non-invasive device for measuring the carbon dioxide concentration in an exhaled breath of a subject. The response values obtained under different relative humidities also show a positive relationship to carbon dioxide concentration, indicating that the non-invasive carbon dioxide sensor according to the disclosure has an excellent resolution capacity in sensing carbon dioxide with stable reproducibility, thereby achieving the purpose of the disclosure.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A non-invasive carbon dioxide sensor, comprising:
a conductive substrate including a base and at least two electrodes disposed on said base and spaced apart from each other;
an electrical transmission layer disposed on said conductive substrate, and including a plurality of carbon nanotubes crossing one another and a plurality of metal oxide nanorods attached to said carbon nanotubes, said carbon nanotubes and said metal oxide nanorods together forming a composite material having a three-dimensional structure; and
a gas sensing layer disposed on said electrical transmission layer, and including a polymer material that contains at least one amino functional group capable of reacting with carbon dioxide.

2. The non-invasive carbon dioxide sensor as claimed in claim 1, wherein said metal oxide nanorods are selected from the group consisting of zinc oxide (ZnO) nanorods, titanium dioxide (TiO₂) nanorods, aluminum oxide (Al₂O₃) nanorods, tricobalt tetroxide (Co₃O₄) nanorods, tin dioxide (SnO₂) nanorods, copper oxide (CuO) nanorods, zirconium dioxide (ZrO₂) nanorods, and combinations thereof.

3. The non-invasive carbon dioxide sensor as claimed in claim 1, wherein said polymer material is selected from the group consisting of polyethylenimine (PEI), polypyrrole (PPy), polyaniline (PANI), and combinations thereof.

4. The non-invasive carbon dioxide sensor as claimed in claim 1, wherein said carbon nanotubes are acid-treated carbon nanotubes.

5. The non-invasive carbon dioxide sensor as claimed in claim 1, wherein said non-invasive carbon dioxide sensor includes X number of said electrical transmission layers, where 10≤X≤90.

6. The non-invasive carbon dioxide sensor as claimed in claim 1, wherein said non-invasive carbon dioxide sensor includes Y number of said gas sensing layers, where 1≤Y≤6.

7. The non-invasive carbon dioxide sensor as claimed in claim 1, wherein said base is selected from the group consisting of a fluorine-doped tin oxide (FTO) substrate, an indium tin oxide (ITO) substrate, a silicon (Si) substrate, a glassy carbon substrate and a metal substrate.

8. The non-invasive carbon dioxide sensor as claimed in claim 1, wherein said base is selected from the group consisting of a polypropylene (PP) base, a polyimide (PI) flexible base, a polyethylene terephthalate (PET) flexible base, a carbon base, and an ultra-thin glass base.

9. The non-invasive carbon dioxide sensor as claimed in claim 1, wherein said three-dimensional structure of said composite material is a hierarchical three-dimensional structure.

10. A method for manufacturing a non-invasive carbon dioxide sensor, comprising:
(a) providing a conductive substrate including a base and at least two electrodes disposed on the base and spaced apart from each other;
(b) heating a composition containing a plurality of carbon nanotubes and a plurality of metal particles so as to transfer the composition into a composite containing a plurality of metal oxide particles attached to the carbon nanotubes;
(c) dispersing the composite obtained in step (b) in a solution containing a metal salt to form a mixture, followed by heating the mixture for a period of time to grow a plurality of metal oxide nanorods using the metal oxide particles attached to the carbon nanotubes as seeds, thereby obtaining an electrical transmission solution containing the carbon nanotubes and the metal oxide nanorods;
(d) heating the electrical transmission solution obtained in step (c);
(e) filtering the electrical transmission solution after step (d), thereby obtaining a composite material that includes the carbon nanotubes and the metal oxide nanorods and that has a three-dimensional structure;
(f) applying the composite material on the conductive substrate to form an electrical transmission layer on the conductive substrate; and
(g) applying a sensing solution on the electrical transmission layer and drying the sensing solution to form a gas sensing layer on the electrical transmission layer, the sensing solution including a polymer material containing at least one amino functional group.

11. The method as claimed in claim 10, further comprising, before step (b), step (b′) of subjecting the carbon nanotubes to an acid treatment, the acid treatment being conducted by adding the carbon nanotubes into an acid solution containing nitric acid and sulfuric acid, and heating the acid solution along with the carbon nanotubes to a temperature ranging from 70° C. to 100° C. for a predetermined time period.

12. The method as claimed in claim 11, wherein, in step (b′), the acid treatment further includes cooling the acid solution along with the carbon nanotubes, rinsing the carbon nanotubes with deionized water, and drying the carbon nanotubes, so as to obtain acid-treated carbon nanotubes.

13. The method as claimed in claim 10, wherein step (g) is conducted repeatedly for several times to form a plurality of the gas sensing layers on the electrical transmission layer.

14. The method as claimed in claim 10, wherein in step (c), the metal salt is selected from the group consisting of a zinc salt, a titanium salt, an aluminum salt, a cobalt salt, a tin salt, a copper salt, and a zirconium salt.

15. The method as claimed in claim 10, wherein in step (g), the polymer material is selected from the group consisting of polyethylenimine (PEI), polypyrrole (PPy), and polyaniline (PANI).

16. The method as claimed in claim 10, wherein in step (c), heating of the mixture is conducted in a water bath.

17. The method as claimed in claim 10, wherein step (e) is conducted by vacuum filtration.

18. The method as claimed in claim 10, wherein, in step (b), the carbon nanotubes are functionalized carbon nanotubes.

19. The method as claimed in claim 18, wherein the functionalized carbon nanotubes have hydroxyl groups or carboxyl groups.

20. The method as claimed in claim 10, wherein, in step (e), the three-dimensional structure of the composite material is a hierarchical three-dimensional structure.
